(19) United States
(12) Patent Application Publication
(10) Pub. No.: US 2015/0342847 A1
(43) Pub. Date: Dec. 3, 2015

(54) NOVEL DERIVATIVES OF SINAPINIC ACID AND THE COSMETIC OR PHARMACEUTICAL USES THEREOF

(71) Applicant: BASF Beauty Care Solutions France SAS, Lyon (FR)

(72) Inventors: Alain DENIS, Villeurbanne (FR); Sabrina LEOTY-OKOMBI, Vaulx Milieu (FR); Delphine RIVAL, Ternay (FR)

(21) Appl. No.: 14/758,007

(22) PCT Filed: Dec. 24, 2013
(86) PCT No.: PCT/FR2013/053260

§ 371 Date: Jun. 26, 2015

(30) Foreign Application Priority Data
Dec. 27, 2012 (FR) 1262848

(51) Int. Cl. A61K 8/37; C07C 69/007; A61Q 19/02; C07C 69/734

(52) U.S. Cl. CPC A61K 8/375; C07C 69/734; C07C 69/007; A61Q 19/02

(57) ABSTRACT

The present invention relates to a compound of general formula (I) below:

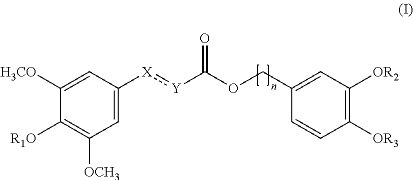

in which:
R₁, R₂ and R₃ independently of one another represent a hydrogen atom; a C1-12 alkyl group; a C2-12 alkenyl group; a C2-12 alkynyl group; a C3-12 cycloalkyl group; a C1-12 acyl group; a sulfonyl group or a phosphonate group; [symbol] represents a CH₂—CH₂ group or a CH═CH group;
n is an integer between 1 and 3;
or a salt thereof.

The invention further relates to a process for synthesizing this compound or salt, to a composition comprising it, to its cosmetic use, more particularly as a depigmenting agent and/or as a radical scavenger, to a cosmetic care process, and to its use as a drug, advantageously intended for preventing and/or treating pathological hyperpigmentation and/or inflammation.

The present invention relates to new sinapic acid derivative compounds and also to uses thereof in cosmetic or pharmaceutical, more particularly dermatological, compositions.

In order to combat solar radiation, the melanocytes in the skin manufacture a dark pigment, melanin, during a process called melanogenesis. One effect of the production of melanin within the skin, however, may be the development of particularly unesthetic pigment spots. In cosmetology, therefore, the search for inhibitors of melanin production has been investigated for a long time. Moreover, although some melanins are in effect protective for the skin, there exists one particular form of melanin, called pheomelanin, which is extremely phototoxic. Agents which inhibit melanin production may therefore prove also to be particularly useful for therapeutic applications, in the context of pathologies characterized by overproduction of melanin. Accordingly there is an ongoing need to identify agents which are capable of acting on melanogenesis, and more particularly on the different pathways of melanogenesis.

Thus, para-coumaric or para-hydroxycinnamic acids have been described in many studies as inhibiting the production of melanin. The reason is thdt these compounds are known to inhibit the enzyme tyrosinase, which is involved in mechanisms of melanogenesis.

Patents JP1013017 (Pola Chemical Industries) and EP1437117 (Cognis France) more particularly describe esterified derivatives of sinapic acid, and also their inhibitory effects on melanogenesis, via inhibition of the enzyme tyrosinase.

Furthermore, in its patent FR2892923, the Applicant company had identified derivatives of para-coumaric acid or para-hydroxycinnamic acid that were of particular interest, exhibiting in particular very good efficacy in the inhibition of melanin production. Therefore these compounds exhibited high anti-tyrosinase activity, and all of the examples were derivatives of cafeic acid, of ferulic acid and of coumaric acid, although no derivative of sinapic acid had been studied and exemplified.

Therefore no document in the prior art describes compounds derived from para coumaric or para-hydroxycinnamic acids that are particularly suitable for topical application, in other words exhibiting acceptable toxicity, being chemically stable, more effectively inhibiting melanin production, and doing so by way of a different pathway, more particularly without involving inhibition of tyrosinase.

No document of the prior art provides compounds derived from sinapic acid which, by acting by a different pathway, might in particular reinforce the effect of an anti-tyrosinase ingredient, preferably synergistically.

Accordingly, the present invention provides a solution to the technical problem of providing new compounds capable of inhibiting melanin production more strongly than those described in the prior art, more particularly those described in patent FR2892923.

Furthermore, there was a particular need to have compounds capable of strongly inhibiting melanin production by acting by way of a pathway other than the tyrosinase inhibition pathway. The present invention likewise solves this technical problem.

The present invention accordingly provides new compounds derived from sinapic acid, of general formula (I), preferably of formula (IIa) and/or of formula (IIb).

A further subject of the invention is the cosmetic and/or pharmaceutical, more particularly dermatological, use of the compounds according to the invention, more particularly as a depigmenting agent and/or as a radical scavenger and/or as an antiinflammatory agent.

The invention also relates to a cosmetic and/or pharmaceutical composition comprising at least one of the compounds according to the invention, and also to the cosmetic and/or pharmaceutical, more particularly dermatological, use thereof.

A further subject of the invention is a process of cosmetic care or of prevention and/or of therapeutic treatment, more particularly dermatological treatment, which comprises the preferably topical application of at least one compound or composition according to the invention.

A final objective of the invention is to provide a process for synthesizing compounds according to the invention.

The present invention accordingly provides new compounds derived from sinapic acid, of general formula (I):

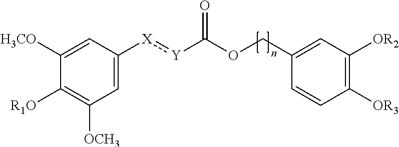

### (I)

in which:
R₂ and R₃ independently of one another represent a hydrogen atom; a C1-12, advantageously C1-6 alkyl group; a C2-12, advantageously C2-6 alkenyl group; a C2-12, advantageously C2-6 alkynyl group; a C3-12, advantageously C3-6 cycloalkyl group; a C1-12, advantageously C1-6 acyl group; a sulfonyl (SO₂H) group or a phosphonate (PO₃H₂) group; [symbol] represents a CH₂—CH₂ group or a CH═CH group;
n is an integer between 1 and 3;
or a salt thereof,
more particularly with the exception of the compound of formula (a) below:

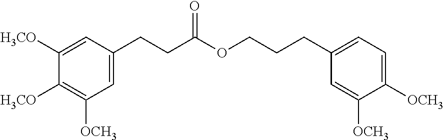 [SMILES: COc1ccc(CCCOC(=O)CCc2cc(OC)c(OC)c(OC)c2)cc1OC]

According to the invention, a “salt” is one of the ionic compounds resulting from the neutralization reaction of the anionic form of the compound according to the invention by a cation, especially an inorganic cation, more particularly sodium, potassium or ammonium. Salts will be formed more particularly by ionic interaction between the cations and the R₁, R₂ and/or R₃ groups in the compounds according to the invention when these groups are selected from: a hydrogen atom; a sulfonyl (SO₂H) group; a phosphonate (PO₃H₂) group. They will then be referred to respectively as sodium, potassium or ammonium salts. The salts according to the invention are preferably sodium and/or potassium and/or ammonium salts, more preferably sodium salts.

The ammonium salts may in particular be in a form in which they are substituted by alkyl groups and/or by organic groups composed of carbon chains. Substituted ammonium salts preferably have four groups including at least one hydrogen atom, the other three being selected independently from: a saturated or unsaturated C1-C2 alkyl chain; a hydrogen atom. According to one preferred embodiment, the groups are all hydrogen atoms. According to an alternative embodiment, the ammonium salts comprise at least one methyl group.

Preferentially, the compounds and salts thereof are preferably cosmetically or pharmaceutically acceptable, more preferably dermatologically acceptable, salts and derivatives, meaning that they are nontoxic for administration to humans, in particular by topical application, and can be applied without risk and without giving rise to allergic or inflammatory reaction more particularly on the skin, the mucosae and/or the keratinous appendages.

According to the invention, “C1-12 alkyl group” means any linear or branched alkyl group containing between 1 and 12 carbon atoms. Mention may be made more particularly of methyl, ethyl, n-propyl, isopropyl, n-butyl, isobutyl, sec-butyl, tert-butyl, n-pentyl, n-hexyl, heptyl, octyl, nonyl and decyl groups. The group in question is advantageously a C1-6 alkyl group, more particularly the methyl group.

According to the invention, “C2-12 alkenyl group” means any linear or branched alkyl group comprising one or more double bonds and containing between 2 and 12 carbon atoms. Mention may be made more particularly of the vinyl group.

According to the invention, “C2-12 alkynyl group” means any linear or branched alkyl group comprising one or more triple bonds and containing between 2 and 12 carbon atoms. Mention may be made more particularly of the ethynyl group.

According to the invention, a “C3-12 cycloalkyl group” means any saturated hydrocarbon ring system containing between 3 and 12 carbon atoms. Mention may be made more particularly of the cyclohexyl group.

According to the invention, a “C1-12 acyl group” means any group R—(C═O) in which R represents a C1-12 alkyl group as defined above. The group in question is advantageously CH₃—C═O.

In one preferred embodiment, the compound according to the invention has the formula (I) in which:
R₁, R₂ and R₃ independently of one another represent a hydrogen atom, a C1-C2 alkyl group, preferably a methyl group; a sulfonyl (SO₂H) group; or a phosphonate (PO₃H₂) group;
or a salt thereof.

Preferably, R₁, R₂ and R₃ are identical and each represents a hydrogen atom.

According to one alternative, the present invention relates to new compounds derived from sinapic acid, of general formula (II)

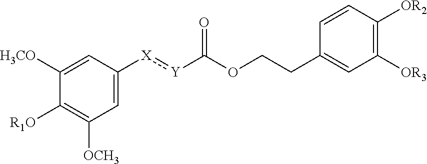

### (II)

in which:
R₁, R₂ and R₃ independently of one another represent a hydrogen atom; a C1-12, advantageously C1-6 alkyl group; a C2-12, advantageously C2-6 alkenyl group; a C2-12, advantageously C2-6 alkynyl group; a C3-12, advantageously C3-6 cycloalkyl group; a C1-12, advantageously C1-6 acyl group; a sulfonyl (SO₂H) group or a phosphonate (PO₃H₂) group; [symbol] represents a CH₂—CH₂ group or a CH═CH group;
or a salt thereof.

In one preferred embodiment, the compound according to the invention has the formula (II) in which:
R₁ is a hydrogen atom and R₂ and R₃ independently of one another represent a hydrogen atom, a C1-C2 alkyl group, preferably a methyl group; a sulfonyl (SO₂H) group; or a phosphonate (PO₃H₂) group; or a salt thereof.

According to one even more preferable embodiment, R₂ and/or R₃ represents a hydrogen atom; preferably R₃ represents a hydrogen atom.

More preferably still, R₁, R₂ and R₃ are identical and each represent a hydrogen atom.

Accordingly, in one preferred embodiment, the compound of the formula (II) according to the invention is hydroxytyrosol dihydrosinapate of formula (IIa):

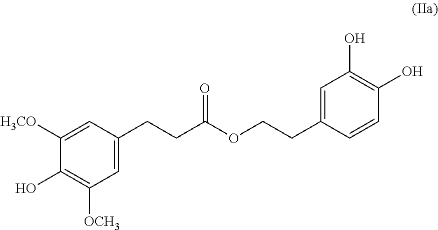 [SMILES: COc1cc(CCC(=O)OCCc2ccc(O)c(O)c2)cc(OC)c1O]

or a salt thereof.

In another preferred embodiment, the compound according to the formula (II) is hydroxytyrosol sinapate of formula (IIb):

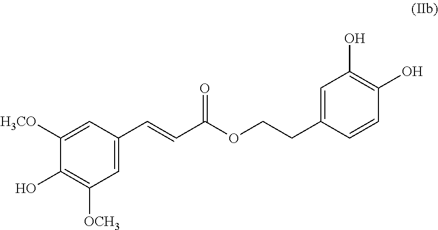 [SMILES: COc1cc(/C=C/C(=O)OCCc2ccc(O)c(O)c2)cc(OC)c1O]

or a salt thereof.

Of course, when [symbol] represents a CH═CH group, the compounds according to the invention include their isomers. They may therefore be trans compounds or else cis compounds, and also a cis/trans mixture. The compounds will preferably be in trans form.

The present invention also provides the cosmetic and/or pharmaceutical, more particularly dermatological, use of at least one of the compounds according to the invention of formula (I), preferably of formula (II), more particularly of formula (IIa) and/or of formula (IIb).

The present invention likewise provides the use of at least one of the compounds according to the invention of formula (I), preferably the compounds of formula (II), more particularly of formula (IIa) and/or of formula (IIb), for the manufacture of a cosmetic composition and/or for the manufacture of a pharmaceutical, more particularly dermatological, composition, and/or as drug.

The reason is, as demonstrated in the examples, that the compounds of formula (I) according to the invention, preferably the compounds of formula (II), and more particularly compounds of formula (IIa) and (IIb), have a very strong inhibitory effect on melanin production. Thus, comparing the compounds of the present invention to the para-coumaric acid derivatives such as those described and exemplified in patent FR2892923, the Applicant company found that the compounds of formula (I) according to the invention had an inhibitory activity on melanin production that was greater than those of the prior art.

Very surprisingly and advantageously, the Applicant company also found that the compounds according to the invention do not inhibit tyrosinase, and this signifies, particularly interestingly, that their pathway of action is different from the compounds described in the prior art and in particular from those exemplified in patent FR2892923, and in a very original way.

“Not inhibit tyrosinase” is understood according to the invention as an activity measured on tyrosinase in the presence of the compound that is not statistically different from that measured in the absence of this compound under the same conditions. The activity of the tyrosinase is measured conventionally, more particularly on human tyrosinase or tyrosinase extracted from melanocytes of murine line B16. The values are not statistically different when the difference is not significant by the student test with p<0.05.

Thus according to one advantageous embodiment, the compounds of formula (I) according to the invention, preferably the compounds of formula (II), more particularly the compound of formula (IIa) and/or the compound of formula (IIb), may be used in combination or in synergy with other depigmenting agents, more particularly anti-tyrosinase agents. This embodiment is of particular advantage to maximize the inhibition of melanin production by acting on the various pathways of melanogenesis. The invention therefore relates to the cosmetic and/or pharmaceutical, more particularly dermatological, use of at least one compound of formula (I) according to the invention, preferably at least one compound of formula (II), more particularly of formula (IIa) and/or (IIb), as a depigmenting agent.

“Depigmenting agent” should be understood to mean a compound capable of reducing the production of melanin.

By “reducing the production of melanin”, is meant, according to the present invention, inhibition of the production of melanin and/or a reduction in the amount of melanin produced, by cells which are capable thereof, more particularly melanocytes. The reduction in melanin production is preferably measured as percentage inhibition of melanin, measured on cells which produce it, for example murine melanocyte cells of line B16, in the presence of the compound to be tested, relative to an untreated control, according to the following formula: percentage inhibition=[(mean stimulated control−mean basal control)−(value sample−mean basal control)]/(mean stimulated control−mean basal control)×100. The stimulated control corresponds to a control treated with conventional agents for stimulating melanin synthesis, such as alpha-MSH, ACTH (adrenocorticotropin hormone), and forskoline. An exemplary embodiment is presented in example B.1. The compounds according to the invention advantageously result in a percentage inhibition of melanin production of at least 40%, preferably at least 50%, more preferably at least 70%. This reduction is measured preferably at a concentration of the test compound according to the invention of 10 μM.

In one preferred embodiment, the present invention further provides the cosmetic and/or pharmaceutical, more particularly dermatological, use of at least one of the compounds according to the invention, preferably the compounds of formula (II), more particularly compounds of formula (IIa) and/or of formula (IIb), for preventing and/or combating hyperpigmentation.

According to the present invention, “hyperpigmentation” means the appearance of pigment spots, which is manifested in at least one darker and/or more colored skin area, and which occurs in particular on accumulation of melanin within said skin area.

These pigment spots are usually benign, but give the skin a particularly unesthetic patchy complexion and/or tone. A number of factors may contribute to their appearance, including, in particular, exposure to the sun, more particularly to UV rays, familial predisposition, aging, more particularly induced by time and/or UV. They may also appear over all parts of the body, more particularly on the male cranium, the back of the hands, on the face, and/or the neckline. Other factors may contribute to the appearance of pigment spots, such as the taking of certain drugs or hormones, as for example in the context of a chloasma (or “pregnancy mask”), which is manifested primarily in pregnant women as the appearance of pigment spots on the neckline, on the neck, more particularly on the face, more particularly on the forehead, the temples and the cheeks. Moreover, pigment spots may appear following aggressive influences such as mechanical aggression and/or aggression by chemical agents or following skin inflammation, for example after skin trauma, eczemic eruptions, psoriatic lesions, or other skin irritations. These pigmentary problems are particularly significant in persons having a so-called non-Caucasian skin type, especially Asian skins, and skins with a phenotype referred to as matt to black, which mark very easily. Hyperpigmentations may also be particularly unesthetic manifestations of pathologies, as in the case, for example, of a hormonal complaint such as Addison's disease, a nutritional deficit complaint or a hepatic insufficiency. In all of these cases, the terminology is “nonpathological hyperpigmentations or pigment spots”.

Accordingly, in one advantageous embodiment, the present invention likewise provides the cosmetic use of at least one of the compounds of formula (I) according to the invention, preferably of at least one compound of formula (II), more particularly of compounds of formula (IIa) and/or of formula (IIb), for preventing and/or combating pigment spots.

The present invention, preferably, accordingly provides the cosmetic use of at least one of the compounds of formula (I) according to the invention, preferably of at least one compound of formula (II), more particularly of compounds of formula (IIa) and/or of formula (IIb), for preventing and/or combating nonpathological hyperpigmentation, more particularly for preventing and/or combating aging-related pigment spots and/or spots induced by exposure to the sun, and/or freckles, and/or postinflammatory spots and/or spots which appear in response to aggressive influences, and/or spots of hormonal origin, more particularly in the context of a chloasma, and/or of drug-related origin, and/or for preventing and/or combating the unesthetic manifestations of a pathology.

In other cases, hyperpigmentation may comprise a true pathology of melanogenesis, which manifests as a deregulation of melanogenesis and hypermelanic melanocytosis. This hypermelanotic pathology may result, for example, from hypersensitization of skin, or may be a purpura, a solar lentigo, a Hon macule, a melanoma, a nevus, a macule, especially nevus of Ota and Hon's nevus, or dermatosis papulosa nigra. These hypermelanotic pathologies are thus called “pathological hyperpigmentation” according to the invention.

Therefore, in another advantageous embodiment, the present invention provides one of the compounds of formula (I) according to the invention, preferably one of the compounds of formula (II), more particularly compounds of formula (IIa) and/or of formula (IIb), for use as a drug, advantageously intended for preventing and/or combating and/or treating pathological hyperpigmentation, more particularly hypermelanotic melanocytosis, especially a purpura, a solar lentigo, a Hon macule, a melanoma, a nevus, a macule, especially nevus of Ota and Hon's nevus and/or dermatosis papulosa nigra.

The compounds of formula (I) according to the invention, as a result of their inhibitory activity on melanin production, are also very suitable for the cosmetic care of skins with normal pigmentation, in other words skins not exhibiting hyperpigmentation, for whitening and/or lightening the skin and/or for increasing the radiance of the complexion, and/or for harmonizing the complexion and/or the tone of the skin. The visible and measurable lightening of the complexion is in effect particularly in persons having a skin of non-Caucasian type, especially Asian skins, and/or skins with a matt to black phenotype.

Moreover, lighter spots which are particularly unesthetic may occur, for example, in the case of post-lesional healing, or as unesthetic manifestations of pathologies such as leucodermas, for example vitiligo. In these latter cases, insofar as it is often difficult to recolor the skin, it may be useful to depigment the residual areas of healthy skin, in order to give the skin as a whole a uniform white shade.

The invention also relates to the cosmetic use of at least one of the compounds of formula (I) according to the invention, preferably of at least one compound of formula (II), more particularly compounds of formula (IIa) and/or of formula (IIb) for whitening the skin, commonly referred to alternatively as “whitening effect”, and/or for lightening the skin, commonly referred to alternatively as “lightening effect”, and/or for enhancing the radiance and/or the luminosity on the skin, commonly referred to alternatively as “brightening effect”, and/or for visibly harmonizing the complexion of the skin.

Very interestingly, the Applicant company has also found that the compounds of formula (I) according to the invention, preferably the compounds of formula (II), more particularly the compounds of formula (IIa) or (IIb), additionally possess radical scavenger activity.

Accordingly, the invention further provides the cosmetic and/or pharmaceutical, more particularly dermatological, use of at least one of the compounds of formula (I) according to the invention, preferably of at least one of the compounds of formula (II), more particularly of the compounds of formula (IIa) and/or (IIb), as a radical scavenger.

According to the invention, the term “radical scavenger” refers to an agent capable of reducing the production of free radicals, also called reactive oxygen species or ROS, capable in particular of capturing free radicals. Radical scavengers are therefore antioxidants.

The term “reducing the production of free radicals” means reducing and/or inhibiting the production and/or the amount of free radicals produced in the skin, especially in the cells, more particularly by the cells and the skin and/or mucosae. Preferentially, the reduction in production of free radicals is measured in vitro, as percentage inhibition relative to a negative control, which is generally the untreated control. Preferentially, the percentage inhibition of DPPH (1,1-diphenyl-2-picrylhydrazyl) radicals will be measured preferably according to a test as described in example B.3a). In this case it has been observed, advantageously, that the compounds of formula (I) according to the invention produce a percentage inhibition of DPPH radicals of at least 50%, preferably at least 80%, relative to the control. According to another test, it is also possible to elect to measure the percentage inhibition of lipid peroxidation, preferably according to a test called TBARS (Thiobarbituric Acid Reactive Substances), especially as described in example B.3b). In this case advantageously, the compounds according to the invention produce a percentage inhibition of lipid peroxidation relative to an untreated control of at least 20%, preferably at least 40%. Preferentially, irrespective of the test selected, this inhibition is measured at a concentration of the test compound according to the invention of 100 μM.

By virtue of their radical scavenger activity, the compounds according to the invention allow oxidative stress to be opposed.

Accordingly, in one advantageous embodiment, the invention relates to the cosmetic use of at least one of the compounds of formula (I) according to the invention, more particularly the compounds of formula (IIa) and/or of formula (IIb), for preventing and/or combating oxidative stress, more particularly as a radical scavenger.

“Oxidative stress” is an imbalance in human beings between the production of free radicals by the cells and the detoxification systems of the skin; in other words, the production of free radicals becomes too great to allow them to be removed. Oxidative stress may be of internal origin, as in the case of the process of skin aging. It may also have an external origin, thus being caused by aggressive agents and/or aggressive conditions. According to the present invention, oxidative stress refers to nonpathological oxidative stress.

Examples of aggressive agents include the following, without limitation: environmental agents such as pollutants, the cigarette, climatic factors (wind, cold, heat), exposure to solar radiation (more particularly UV rays), emotional factors, especially emotional stress, and/or chemical agents such as heavy metals, detergents, compounds present in cosmetic treatments, such as fragrances, preservatives, pH, alcohols, AHAs, or dermatological agents such as vitamin A acid. Oxidative stress caused by solar radiation, in particular, is one of the mechanisms involved in a particular and complex phenomenon of aging: light-induced aging, also called “photo-aging”.

Examples of aggressive conditions include the following, without limitation: perspiration, and mechanical aggressions such as epilation, shaving, and rubbing.

In all of these cases, the manifestations caused by oxidative stress that are benign, in other words non-pathological and purely esthetic and/or uncomfortable, are usually manifested:
on or in the skin, especially by accelerated aging of the skin, with more particularly a dull and/or grey complexion, and/or a patchy complexion, and/or a loss of radiance and/or of transparency of the skin, and/or the premature formation of wrinkles or fine lines, and/or a loss of softness, and/or of suppleness and/or elasticity of the skin, and/or the appearance of pigment spots, given that free radicals are capable of oxidizing derivatives of DOPA (dihydroxyphenylalanine), which are involved in melanogenesis;
on or in the keratinous appendages, more particularly the hair, and/or nails, and/or eyelashes, in particular as a reduction in the vigor of the hair and/or an alteration in its appearance, and more particularly a dull aspect.

The compounds of formula (I) according to the present invention, owing to the properties thereof as radical scavengers and of inhibition of the release of prostaglandins, are particularly suitable for the realization of calmative care and/or care of sensitive skins. They may therefore be applied to any type of skin, and particularly for the care and/or cosmetic treatment of sensitive skins, and/or reactive and/or hyperreactive skins and/or skins which have been temporarily rendered sensitive. Generally speaking, sensitive skins may be defined as skins which no longer tolerate, or tolerate to a very low extent, the aggressive agents and/or aggressive conditions. Sensitive skins or skins which have been rendered sensitive temporarily are not skins having pathological characters. They may nevertheless react to the aggressive agents and/or conditions, with unesthetic and/or uncomfortable skin and/or mucosal manifestations.

According to the invention, “unesthetic and/or uncomfortable skin and/or mucosal manifestations” are nonpathological manifestations such as the following: stinging sensation, and/or burning sensation, and/or itching sensation, and/or tautness, and/or redness, and/or visible squama, and/or thickening of the skin. Accordingly, the “sensitive skin” character may be estimated by the subject themself with skin sensations, or established objectively by a dermatologist.

The invention thus relates to the use of at least one compound of formula (I) according to the invention, preferably of at least one compound of formula (II), more particularly of the compound of formula (IIa) and/or of formula (IIb), for preventing and/or combating the benign manifestations caused by oxidative stress, preferably in or on the skin and/or keratinous appendages, and/or for preventing and/or combating skin and/or mucosal aging, and/or solar radiation and/or light-induced skin and/or mucosal aging, and/or the unesthetic and/or uncomfortable skin and/or mucosal manifestations, preferably caused by aggressive agents and/or aggressive conditions, more particularly caused by solar radiation.

The invention thus preferably relates to the use of at least one compound of formula (I) according to the invention, preferably of at least one compound of formula (II), more particularly of the compound of formula (IIa) and/or of formula (IIb), for preventing and/or combating skin aging, more particularly light-induced skin and/or mucosal aging, more particularly for preventing and/or combating at least one condition selected from the following: a dull and/or patchy complexion, transparency of the skin and/or thinning thereof and/or a loss of radiance, and/or a loss of softness, and/or a loss of suppleness, and/or a loss of elasticity, and/or formation of wrinkles and/or fine lines, and/or appearance of pigment spots.

Furthermore, the compounds of formula (I) according to the invention are antiinflammatory agents and more particularly inhibitors of the release of prostaglandin, especially E2.

Accordingly, in another advantageous embodiment, the present invention provides one of the compounds of formula (I) according to the invention, preferably one of the compounds of formula (II), more particularly compounds of formula (IIa) and/or (IIb), for use as an antiinflammatory drug, more particularly an inhibitor of the release of prostaglandins, more particularly E2, more particularly for use as a drug intended for preventing and/or treating inflammation.

An “antiinflammatory agent” according to the invention means an agent capable of preventing and/or treating inflammation. These inflammations are preferably skin and/or mucosal inflammations, especially those associated with eczema, and/or psoriasis, and/or dermatitis, and/or urticaria, and/or rosacea, and/or acne, and/or solar erythema, and/or hypertrophic scars, and/or keloids.

A “prostaglandin release inhibitor” is an agent which prevents the release and/or reduces the amount of prostaglandin in the skin and/or the mucosae, more particularly prostaglandins E2.

The compounds of formula of formula (I) according to the invention, preferably the compounds of formula (II), more particularly the compounds of formula (IIa) and/or of formula (IIb), are preferably used for topical application, preferably for the skin of the hands, and/or of the face and/or of the neckline, and/or of the cranium, more particularly of the male cranium, and/or for the nails, and/or the hair.

The compounds of formula of formula (I) according to the invention, preferably the compounds of formula (II), more particularly the compounds of formula (IIa) and/or of formula (IIb), are preferably used at a concentration of from 1×10⁻⁶ to 1×10⁻¹ M, preferably 1×10⁻⁵ to 1×10⁻² M, more preferably 1×10⁻⁴ M.

The compounds of formula (I) according to the invention, preferably the compounds of formula (II), more particularly the compounds of formula (IIa) and/or of formula (Mb), may be used on their own or in the form of a cosmetic and/or a pharmaceutical, more particularly dermatological, composition.

The invention accordingly likewise provides a composition, more particularly a cosmetic and/or pharmaceutical, more particularly dermatological composition, comprising at least one of the compounds of formula (I) according to the invention, preferably the compounds of formula (II), more particularly the compounds of formula (IIa) and/or of formula (IIb), preferably with an acceptable cosmetic and/or pharmaceutical, and preferably dermatological, vehicle.

The cosmetic or pharmaceutical compositions according to the invention may include an excipient such as, for example, at least one compound selected from the group consisting of preservatives, emollients, emulsifiers, surfactants, moisturizers, thickeners, conditioners, matting agents, stabilizers, antioxidants, texture agents, shine agents, film formers, solubilizers, pigments, dyes, fragrances, and sunscreens.

These excipients are preferably selected from the group consisting of amino acids and derivatives thereof, polyglycerols, esters, polymers, and derivatives of cellulose, lanolin derivatives, phospholipids, lactoferrins, lactoperoxidases, sucrose-based stabilizers, E vitamins and derivatives thereof, natural and synthetic waxes, vegetable oils, triglycerides, unsaponifiables, phytosterols, plant esters, silicones and derivatives thereof, protein hydrolysates, jojoba oil and derivatives thereof, fat/water-soluble esters, betaines, amine oxides, plant extracts, saccharose esters, titanium dioxides, glycines, and parabens, and more preferably from the group consisting of butylene glycol, steareth-2, steareth-21, glycol-15 stearyl ether, cetearyl alcohol, phenoxyethanol, methylparaben, ethylparaben, propylparaben, butylparaben, natural tocopherols, glycerin, dihydroxycetyl sodium phosphate, isopropyl hydroxycetyl ether, glycol stearate, triisononanoin, octyl cocoate, polyacrylamide, isoparaffin, laureth-7, a carbomer, propylene glycol, glycerol, bisabolol, a dimethicone, sodium hydroxide, PEG 30-dipolyhydroxystearate, capric/caprylic triglycerides, cetearyl octanoate, dibutyl adipate, grapeseed oil, jojoba oil, magnesium sulfate, EDTA, a cyclomethicone, xanthan gum, citric acid, sodium lauryl sulfate, mineral waxes and mineral oils, isostearyl isostearate, propylene glycol dipelargonate, propylene glycol isostearate, PEG 8, beeswax, hydrogenated palm kernel oil glycerides, hydrogenated palm oil glycerides, lanolin oil, sesame oil, cetyl lactate, lanolin alcohol, castor oil, titanium dioxide, lactose, saccharose, low-density polyethylene, and isotonic saline solution.

The aforementioned compositions are advantageously formulated in a form selected from the group consisting of an aqueous or oily solution, a cream or a gel which is aqueous, or an oily gel, especially in a pot or in a tube, more particularly a shower gel, a shampoo; a milk; an emulsion, a microemulsion or a nanoemulsion, more particularly oil-in-water or water-in-oil or multiple or silicone-containing; a mask; a lotion, especially in a glass or plastic bottle or in a measuring bottle or as an aerosol; an ampoule; a liquid soap; a dermatological bar; an ointment; a mousse or foam; an anhydrous product, preferably liquid, pasty or solid, in the form for example of a stick, especially in the form of lipstick or of tablets.

According to the present invention, “topical application” means the application of the composition to the surface of the skin and/or to the mucosae and/or to the keratinous appendages.

The terms “acceptable cosmetic or dermatological vehicle” used here signify that the composition or its components are suitable for use in contact with the human skin, without undue toxicity, incompatibility, instability, allergic response, or equivalents thereof.

The terms “acceptable pharmaceutical vehicle” used here signify that the composition or its components are suitable for use in contact with at least part of the human body, without undue toxicity, incompatibility, instability, allergic response, or equivalents thereof.

Numerous cosmetically active ingredients are known to the skilled person for enhancing the health and/or the physical appearance of the skin. The skilled person knows how to formulate cosmetic or dermatological compositions in order to obtain the best effects. Moreover, the compounds described in the present invention may have a synergistic effect when combined with one another. These combinations are likewise covered by the present invention.

The CTFA Cosmetic Ingredient Handbook, Second Edition (1992) describes various cosmetic and pharmaceutical ingredients which are commonly used in the cosmetic and pharmaceutical industry, and which more particularly are suitable for topical use. Examples of these classes of ingredients include, without being limited thereto, the following compounds: abrasive, absorbents, compound for esthetic purposes such as fragrances; pigments; dyes; essential oils; astringents such as clove oil, menthol, camphor, eucalyptus oil, eugenol, menthyl lactate, witch hazel distillate; anti-acne agents; antiflocculants; antifoams; antimicrobial agents such as iodopropyl butylcarbamate; antioxidants such as ascorbic acid; binders; buffers; swelling agents; chelating agents; additives; biocides; denaturing agents; thickeners; and vitamins; film-forming materials; polymers; opacifiers; pH modifiers; reducing agents; conditioning agents such as humectants, and derivatives or equivalents thereof.

In one advantageous embodiment, the compositions according to the invention comprise one of the compounds of formula (I) according to the invention or a mixture thereof, preferably one of the compounds of formula (II) or a mixture thereof, more particularly the compound of formula (IIa) and/or of formula (IIb), in which the compound according to the invention or the mixture is present at concentrations of from 1×10⁻⁶ to 1×10⁻¹ M, preferably 1×10⁻⁵ to 1×10⁻² M, more preferably 1×10⁻⁴ M.

In one advantageous embodiment, the cosmetic or pharmaceutical compositions according to the invention include other ingredients of interest, particularly of cosmetic, pharmaceutical or dermatological interest, preferably ingredients having complementary properties. These are preferably depigmenting ingredients and/or ingredients which improve the brightness of the complexion, preferably tyrosinase inhibitors, and/or radical scavenger ingredients, and/or antiinflammatory ingredients. The complementary ingredients preferably act in synergy with the latter ingredients, to provide a more effective cosmetic composition.

According to one advantageous embodiment, therefore, the cosmetic compositions according to the invention include at least one other ingredient selected from the group consisting of:
a depigmenting agent, as for example derivatives of para-coumaric acid and para-hydroxycinnamic acid, such as caffeic acid, sinapic acid, ferulic acid, and also those described by the Applicant company in patent FR2892923, an extract of Juniperus communis or of Galeapsis ochroleuca, such as those described by the Applicant company in patent application WO 2008/148892, kojic acid, hydroquinone, α- and β-arbutin, other glycosidic hydroquinones, deoxyarbutin, diacetyl-boldine, azelaic acid, octadecenedioic acid, linoleic acid, conjugated linoleic acid, α-lipoic acid, glutathione and derivatives thereof, undecylenoyl-phenylalanine, vitamin C and derivatives thereof such as magnesium L-ascorbyl-phosphate, niacinamide, 4-n-butyl-resorcinol, α- and β-hydroxy acids, ellagic acid, resveratrol, Morus alba extracts, extracts of glabridine and of liquorice, imperatorin and isoimperatorin, Angelica dahurica extracts, extracts of yarrow and of centaureidin, Bettis perennis extracts, Phyllanthus emblica extracts, extracts of watercress, Veratum nigrum extracts, Sophora flavescens extracts, melanin-degrading enzymes derived from ascomycetes;
an antiinflammatory agent which in particular inhibits PLA2, and more particularly one of the active agents described in patent application FR2847267, preferably a Pueraria lobata root extract (Inhipase®);
a radical scavenger or antioxidant such as, for example, radical scavengers such as those described by the Applicant company in patent FR2892923, tocopherol (vitamin E) or derivatives thereof, vitamin C or derivatives thereof, carotenoid, ubiquinone, green tea;
a sunscreen, as for example metal oxide pigments, dibenzoylmethane derivatives, anthranilates, cinnamic derivatives other than those of formula (I), salicylic derivatives, camphor derivatives, benzophenone derivatives, triazine derivatives, benzylmalonate derivatives, benzimidazole derivatives, imidazolines, derivatives of p-aminobenzoic acid (PABA), of benzotriazole, of methylenebis(hydroxyphenylbenzotriazole), of benzoxazole, polymers filter and silicones filter, dimers derived from α-alkylstyrene, 4,4-diarylbutadienes, merocyanin derivatives, and indanylidene filter;
an agent which stimulates synthesis of fibronectin, more particularly a corn extract, an extract of this kind being sold in particular by the Applicant company under the name Definer™;
an agent which mimics the effects of DHEA, especially stimulation of lipid synthesis, prevention of glycation, more particularly an extract of mallow leaves (Malva sylvestris) sold by the Applicant company under the name Phystrogene™;
a moisturizing agent, especially an agent which stimulates lipid synthesis, as for example a biotechnologically modified yeast extract sold by the Applicant company under the name of Relipidium™;
an agent for restoring dermal structure, such as an ursolic acid stabilized in a liposome, which is sold by the Applicant company under the name of Ursolisome™;
an antiglycation agent, more particularly those described in patent application WO2009007411, preferably Davila rugosa extract;
an agent which stimulates synthesis of laminin, more particularly a biotechnologically modified malt extract, an extract of this kind being sold in particular by the Applicant company under the name Basaline™;
an agent which stimulates the expression and/or activity of hyaluronane synthase 2 (HAS2), such as the plant extracts described in patent application FR2 893 252 A1, and more particularly an aqueous extract of galanga (Alpinia galanga);
an agent which stimulates the synthesis of molecules of the extracellular matrix, more particularly glycoaminoglycans (GAG) and/or elastin and/or collagen, such as, for example, retinol, vitamin C and derivatives thereof, tetrapeptides containing between 50 to 500 ppm of palmitoyl-Gly-Gln-pro-Arg, such as those sold under the name Matrixyl™ by Sederma, or a mixture of plant extracts which are sold under the name Strivectin™, or arabinogalactan or compounds containing it;
an agent which mimics the effects of beta-endorphins, such as those cited in patent application US 2006069032, a cocoa extract, or an extract of Tephrosia purpurea (Solliance);
an agent which protects fibroblast growth factor (FGF), more particularly FGF2, such as the plant extract described in patent application GB244036 in the name of the Applicant company, more particularly an extract of Hibiscus abelmoscus, especially that sold under the name Linefactor™;
an agent which stimulates the activity and/or proliferation of fibroblasts, such as an extract of fermented soy peptide which is sold under the name Phytokine™, optionally in combination with an extract of Hibiscus abelmoscus sold under the name Linefactor™, as described in patent application WO2009121422 in the name of the Applicant company;
an agent which stimulates the activity and/or the synthesis of Lysyl Oxidase Like LOXL, especially selected from the compounds described in patent application FR2855968, more particularly an extract of dill (Peucedanum graveolens) for the stimulation of the elastic fibers;
a draining agent, especially hesperitin laurate (Flavagrum®), or quercetin caprylate (Flavenger®);
a veinotonic agent such as, for example, an agent which traps spermine and/or spermidine, more particularly a kappa-carrageenan hydrolysate such as that described in patent application WO2009000935;
an agent which lowers the threshold of sensitivity of the immune response, such as, for example, a plant extract of Cestrum latifolium such as that described in patent application WO2009112590 and sold under the name Symbiocell™;

and also mixtures thereof.

The invention therefore further relates to a cosmetic composition and/or a pharmaceutical composition, more particularly a dermatological composition, comprising at least one of the compounds according to the invention, preferably the compounds of formula (IIa) and/or (IIb), in an acceptable cosmetic and/or pharmaceutical and/or dermatological vehicle, optionally in combination with at least one other ingredient of interest, in particular of cosmetic, and/or pharmaceutical, preferably dermatological, interest, preferably as defined above.

The cosmetic compositions according to the invention are, in particular, anti-age care compositions, and/or anti-wrinkle and/or day care compositions, and/or sun protection care and/or after-sun and/or depigmenting care compositions, and/or calmative care compositions.

The cosmetic or pharmaceutical composition according to the invention is preferably intended for topical application.

More particularly, it is intended for the skin of the hands, and/or of the face and/or of the neckline, and/or of the cranium, more particularly of the male cranium, and/or for the nails, and/or the hair.

The present invention also provides a process for cosmetic care or of prevention and/or therapeutic, more particularly dermatological, treatment, comprising the administration, preferably by topical application, of at least one compound of formula (I) according to the invention, preferably of at least one compound of formula (II), more particularly of the compound of formula (IIa) and/or of formula (IIb), optionally in the form of a composition according to the invention, preferably for one or other of the aforementioned uses.

The process according to the invention is advantageously intended for human beings.

In one advantageous embodiment, the process for cosmetic care or prevention and/or therapeutic treatment according to the invention is a process for reducing the production of melanin, more particularly in or on the skin.

The present invention advantageously provides a cosmetic care process for preventing and/or combating pigment spots, comprising the topical application, more particularly to these pigment spots, of at least one compound of formula (I) according to the invention, preferably of at least one compound of formula (II), more particularly the compound of formula (IIa) and/or of formula (IIb), optionally in the form of a cosmetic composition according to the invention.

The invention also embraces a cosmetic care process for whitening the skin, also called “whitening effect”, and/or for lightening the skin, alternatively called “lightening effect”, and/or for imparting brightness and/or luminosity to the skin, alternatively called “brightening effect”, and/or for harmonizing the appearance of the skin, in particular of skin areas with normal pigmentation, preferably for matt to brown skins.

In another advantageous embodiment, the process for cosmetic care or prevention and/or therapeutic treatment according to the invention is a process for reducing the production of free radicals, more particularly in or on the skin and/or the mucosae and/or the keratinous appendages, more particularly the hair, the eyelashes, the nails and/or eyebrows.

The invention also relates advantageously to a cosmetic care process for preventing and/or combating oxidative stress, advantageously for preventing and/or combating the benign manifestations caused by free radicals in or on the skin and/or keratinous appendages, more particularly the hair, the eyelashes, the nails and/or eyebrows, and/or for preventing and/or combating the unesthetic and/or uncomfortable skin and/or mucosal manifestations.

The invention further provides a process of therapeutic, preferably dermatological, treatment and/or prevention of pathological hyperpigmentation, and/or of inflammation, which comprises the administration, preferably by topical application, of at least one compound of formula (I) according to the invention, preferably of at least one compound of formula (II), more particularly of compounds of formula (IIa) and/or of formula (IIb), optionally in the form of a pharmaceutical, more particularly dermatological, composition according to the invention.

The compounds according to the invention, more particularly the compounds of formula (IIa) and/or of formula (IIb), optionally in the form of a composition according to the invention, are preferably applied daily, preferentially one to two times per day, preferentially in the morning and/or evening.

The compounds according to the invention, more particularly the compounds of formula (IIa) and/or of formula (IIb), optionally in the form of a composition according to the invention, are preferably applied to the skin of the hands, and/or of the face and/or of the neckline, and/or of the cranium, more particularly of the male cranium, and/or to the nails, and/or, the hair.

The compounds according to the invention, more particularly the compounds of formula (IIa) and/or of formula (IIb), optionally in the form of a composition according to the invention, are preferably applied at a concentration of from 1×10⁻⁶ to 1×10⁻¹ M, preferably 1×10⁻⁵ to 1×10⁻² M, more preferably 1×10⁻⁴ M.

The present invention also provides a process for synthesizing compounds of formula (I) according to the invention, more particularly compounds of formula (IIa) and/or of formula (IIb) or a salt thereof, and also their isomers.

Advantageously, the process for synthesizing compounds of formula (I) according to the invention, more particularly of compounds of formula (IIa) and/or of formula (IIb), comprises a step of coupling an acid with an alcohol, advantageously by acid catalysis.

For the synthesis of compounds of formula (I) according to the invention, the acid is therefore preferably of formula: IIIa

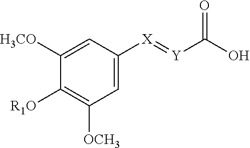

in which, X, Y and R1 have the same meaning as in the formula (I)
and the alcohol is therefore preferably of formula: IIIb

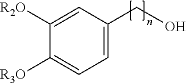

in which n, R2 and R3 have the same meaning as in the formula (I).

For the synthesis of compounds of formula II, the acid is preferably of formula: IVa

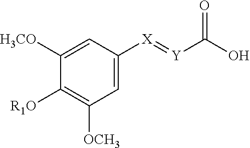

in which, X, Y and R1 have the same meaning as in the formula (I)
and the alcohol is preferably of formula: IVb

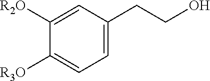

in which n, R2 and R3 have the same meaning as in the formula (I).

For the synthesis of compounds of formula IIa), the acid is preferably of formula: Va

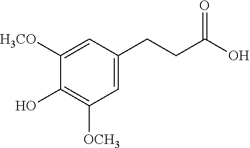 [SMILES: COc1cc(CCC(=O)O)cc(OC)c1O]

and the alcohol is preferably of formula: Vb

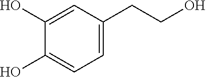 [SMILES: OCCc1ccc(O)c(O)c1]

For the synthesis of compounds of formula IIb), the acid is preferably of formula: VIa

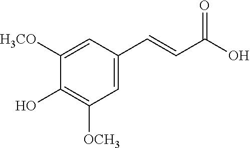 [SMILES: COc1cc(/C=C/C(=O)O)cc(OC)c1O]

and the alcohol is preferably of formula: VIb

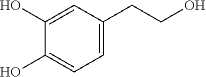 [SMILES: OCCc1ccc(O)c(O)c1]

The acid for coupling is therefore selected preferably from sinapic acid, especially for the synthesis of compounds of formula (I) in which [symbol] represents a CH═CH group, more particularly the compound of formula (IIb), and/or dihydrosinapic acid, especially for the synthesis of compounds of formula (I) in which [symbol] represents a CH₂—CH₂ group, more particularly for the synthesis of the compound of formula (IIa).

Sinapic acid is readily available commercially, but may also be extracted from plants containing it, as for example sunflower (Helianthus annuus), wheat (Triticum vulgare), chinese cabbage (Brassica rapa) or Polygala tenuifollia.

For the synthesis of dihydrosinapic acid, sinapic acid will be preferably selected as starting compound. In this case the dihydrosinapic acid will be synthesized in situ, in other words by a succession of reactions carried out in the same reactor. This synthesis is preferably performed by hydrogenation of the double bond of sinapic acid, preferably in the presence of Pd—C (palladium on carbon), preferentially of 5% Pd—C. This embodiment is particularly advantageous for the synthesis of the compound of formula (IIa).

The invention therefore likewise provides a process for synthesizing a compound of formula (I) in which n=2. In this case, the alcohol for coupling is preferably hydroxytyrosol.

Hydroxytyrosol may easily be found in commerce, or may be extracted from plants, as for example olive (Olea europea). Owing to its instability, however, hydroxytyrosol is preferably synthesized in situ from dihydroxyphenylacetic acid, which is likewise available commercially.

Accordingly, the process for synthesis of compounds of formula (I) according to the invention, preferably of compounds of formula (II), more particularly of compounds of formula (IIa) and/or of formula (IIb), preferably comprises a step of synthesis of hydroxytyrosol in situ, preferably starting from dihydroxyphenylacetic acid, preferably by conversion of the dihydroxyphenylacetic acid to methyl ester, preferably by acid catalysis, preferentially using para-toluenesulfonic acid.

The methyl ester thus obtained is preferably reduced subsequently to alcohol to form hydroxytyrosol.

When the acid for the coupling reaction is dihydrosinapic acid, this step of in situ synthesis of hydroxytyrosol is performed advantageously in parallel with the in situ synthesis of dihydrosinapic acid.

For the synthesis of a compound according to the invention of formula (I) for which n is not 2, hydroxytyrosol will be replaced by another compound for the coupling reaction. For example and with preference, it will be possible to select 4-(hydroxymethyl)benzene-1,2-diol (UICPA name) as alcohol for coupling when n=1, and 4-(3-hydroxypropyl)benzene-1,2-diol (UICPA name) for example when n=3.

Starting compounds of these kinds may readily be found commercially.

According to a preferential embodiment, the invention provides a process for synthesizing the compound of formula (IIb) or a salt thereof that comprises at least the steps of:
a) synthesizing, preferably in situ, hydroxytyrosol from dihydroxyphenylacetic acid;
b) coupling the hydroxytyrosol obtained in step a) with sinapic acid.

According to a preferential embodiment of the invention, the process for synthesizing the compound of formula (IIa) or a salt thereof comprises at least the steps of:
a) synthesizing, preferably in situ, hydroxytyrosol from dihydroxyphenylacetic acid;
b) synthesizing, preferably in situ, dihydrosinapic acid from sinapic acid;
c) coupling the hydroxytyrosol obtained in step a) with the dihydrosinapic acid obtained in step b).

Steps a) and b) are advantageously performed in parallel.

When groups R₁, R₂ and/or R₃ of the compounds to be synthesized according to the invention are not hydrogen atoms, the same alcohols or acids will be used as starting reactant, after having been alkylated, sulfated and/or phosphated and/or esterified, depending on the nature of the respective group R, and according to conventional techniques. Alternatively, it will be possible for this alkylation, sulfation, phosphation and/or esterification reaction to be carried out after the coupling reaction in the same way. Advantageously, when the compound comprises an alkyl group in position R₁, R₂ and/or R₃, the alkylation reaction will be carried out before the coupling reaction. The compounds thus obtained are preferably compounds of formula (IIa) and/or of formula (IIb).

The invention likewise provides the compounds obtained by the synthesis processes according to the invention.

The examples hereinbelow form an integral part of the present invention and serve to illustrate it, but without constituting any limitation. Any feature appearing to be novel over any prior art, on the basis of the description taken as a whole, including the examples, forms an integral part of the invention with respect to its function and to its generality.

Moreover, in the examples, all of the percentages are given by weight, unless indicated otherwise, and the temperature is expressed in degrees Celsius, unless indicated otherwise, and the pressure is atmospheric pressure, unless indicated otherwise.

### EXAMPLES

### A. Synthesis of the Compounds According to the Invention

### Example A.1

### Synthesis of Hydroxytyrosol Sinapate of Formula (IIb) According to the Invention

Step a): Synthesis In Situ of Hydroxytyrosol from Dihydroxyphenylacetic Acid

Dihydroxyphenylacetic acid is dissolved in methanol and esterified at reflux over 3 hours with catalysis by para-toluenesulfonic acid, in the absence of light. The methanol is evaporated and methyltetrahydrofuran (methylTHF) is added. The resulting solution is washed with sodium bicarbonate and with brine. Following evaporation of the solvent, the residue is taken up in tetrahydrofuran and lithium aluminum hydride is added to the mixture. The whole mixture is refluxed for 2 hours. 5N HCl solution is added to the mixture, which is subsequently extracted with methylTHF.

Step b): Coupling of the Hydroxytyrosol Obtained in Step a) with Sinapic Acid.

The solution of hydroxytyrosol in toluene that was obtained in step a), along with 4% of para-toluenesulfonic acid, are added to sinapic acid. The reaction mixture is refluxed for 12 hours. Following filtration to remove the salts and washing of the organic phase, the reaction mixture is concentrated to dryness under vacuum.

### Example A.2

### Synthesis of Hydroxytyrosol Dihydrosinapate of Formula (IIa) According to the Invention

Step a): Synthesis In Situ of Hydroxytyrosol from Dihydroxyphenylacetic Acid.

Hydroxytyrosol is synthesized by the protocol described in step a) of example A.1. above.

Step b): Synthesis In Situ of Dihydrosinapic Acid from Sinapic Acid.

In parallel with the synthesis of hydroxytyrosol in situ as defined in step a), the double bond of sinapic acid is reduced over 4 hours under 5 bar of hydrogen at ambient temperature in the presence of Pd—C (5%) in methylTHF. Following filtration over Clarcel, the solution is introduced directly into step c).

Step c): Coupling the Hydroxytyrosol Obtained in Step a) with the Dihydrosinapic Acid Obtained in Step b).

The coupling reaction is carried out according to the protocol defined in step b) of example A.1. above.

### B. Results of Experimental Tests

In the examples below, reference is made to a prior-art ferulic acid derivative which corresponds to example 1 of Table 11 in patent FR2892923. This compound is N-trans-feruloyldopamine, of formula:

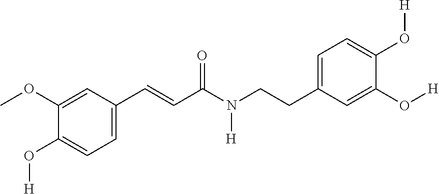 [SMILES: COc1cc(/C=C/C(=O)NCCc2ccc(O)c(O)c2)ccc1O]

### Example B.1

### Tests of Inhibition of Melanin Production

Principle:

Determination of the percentage inhibition of melanin production by the hydroxytyrosol dihydrosinapate of the invention of formula (IIa) or the ferulic acid derivative of the prior art, by quantitative analysis of the total melanin synthesized by melanocytes of murine line B16 following application of the compound under test.

Protocol:

Melanocytes (B16 cells) are cultured for 24 hours in a 96-well plate in an appropriate medium. The culture medium is withdrawn and then replaced with medium supplemented or not with an α-MSH derivative containing either the compounds under test or the negative control (culture medium and solvent), or the positive control (kojic acid). After 72 hours of incubation, the culture medium is withdrawn, and total melanin is quantified by measuring the absorbance at 405 nm against a standard range of synthetic melanin from 0.78 to 100 μg/mL.

The positive experimental control is kojic acid at a concentration of 0.025 mg/mL, with which the percentage inhibition of melanin production was 50%.

Results:

The results are expressed as percentage inhibition of melanin production relative to the negative control, and are presented in Table 1.

The inhibitory activity on melanin production of the product tested is calculated as a percentage according to the following formula:

% inhibition=[(mean stimulated control−mean basal control)−(sample value−mean basal control)]/(mean stimulated control−mean basal control)×100

where:
Mean stimulated control: mean of the values obtained with a medium supplemented with an alpha-MSH derivative
Sample value: value obtained with the compound under test
Mean basal control: Mean of the values obtained with a medium not supplemented with an alpha-MSH derivative

TABLE 1
Percentage inhibitions of melanin in B16 cells after application
of the hydroxytyrosol dihydrosinapate according to the invention
or of a prior-art ferulic acid derivative.

| | Concentration | % inhibition of melanin +/− |
|---|---|---|
| Sample | tested | standard deviation |
| Hydroxytyrosol | 10 μM | 75 +/− 1 |
| dihydrosinapate (IIa) | 30 μM | 106 +/− 1 |
| | 50 μM | 115 +/− 1 |
| Prior-art ferulic acid | 10 μM | 37 +/− 4 |
| derivative | 30 μM | 76 +/− 2 |
| | 50 μM | 88 +/− 2 |

### Conclusions

These results show that hydroxytyrosol dihydrosinapate (IIa) has a strong inhibitory effect on the production of melanin, even at low concentrations. Indeed, a substantial inhibitory effect of 75% is obtained for the smallest concentration tested (10 μM).

These results demonstrate effectively the advantages of the compounds according to the invention and their better efficacy relative to the compounds of the prior art.

### Example B.2

### Tests of Anti-Human Tyrosinase Activity

Principle:

Study of the inhibition of tyrosinase by the hydroxytyrosol dihydrosinapate of the invention of formula (IIa), in comparison with the prior-art ferulic acid derivative, by quantitative analysis of the human tyrosinase activity of melanocytes cultured as a monolayer following application of the compound under test.

Protocol:

Normal human melanocytes (obtained from abdominal plasty) are seeded in a 24-well plate at 80000 cells per well. They are cultured until confluence, and the compounds under test are applied in the culture media for 24 hours. After 24 hours, the media are removed and the melanocytes are detached by mechanical action. Extraction is carried out by thermal shock and then the supernatants are recovered and incubated with MBTH (methyl benzothiazolinone-hydrazone) (Sigma) and L-Dopa (Sigma). The optical density (OD) at 490 nm (OD₄₉₀) is measured after 30 minutes of incubation, and the inhibition of tyrosinase is calculated according to the following formula:

% inhibition=100−[100×(OD₄₉₀ sample/level of proteins in sample)/(OD₄₉₀ negative control/level of proteins in negative control)]

The OD₄₉₀ is therefore related to the level of protein, which is the measurement in each culture well. A percentage anti-tyrosinase activity is therefore calculated relative to the negative control, which is the untreated control.

The positive experimental controls are kojic acid and arbutin at 1 mM, and the expected inhibition is approximately 35%, thereby allowing the test to be validated.

Results:

The results are presented as % inhibition of activity of tyrosinase, in Table 2.

TABLE 2
Inhibition of human tyrosinase after application of the
hydroxytyrosol dihydrosinapate according to the invention
or of a prior-art ferulic acid derivative

| | | | % inhibition of activity of |
|---|---|---|---|
| | | | tyrosinase +/− standard |
| Sample | Concentration tested | | deviation |
| Hydroxytyrosol | 1 | μM | −5.85 +/− 9.76 |
| dihydrosinapate (IIa) | 10 | μM | −4.53 +/− 5.17 |
| | 100 | μM | −36.64 +/− 29 |
| Prior-art ferulic acid | 1 | μM | −9.22 +/− 99.66 |
| derivative | 10 | μM | 18.82 +/− 6.63 |
| | 100 | μM | 67.85 +/− 3.39 |
| Kojic acid | 0.1% (in M) | | 20% ± 5% |
| Arbutin | 1 | mM | 65% ± 8.71% |

Conclusions:

These results show that the hydroxytyrosol dihydrosinapate (IIa) according to the invention does not inhibit the activity of the human tyrosinase in the melanocytes, in contrast to what had already been observed for the ferulic acid derivatives of the prior art.

This suggests that the inhibitory effect on the production of melanin by the compounds according to the invention, especially as observed in example B.1. above, is exerted by way of different pathways from those of the compounds already known.

### Example B.3

### Tests of Radical Scavenger Activity

### Example B.3a

Study by DPPH Test (1,1-diphenyl-2-picrylhydrazyl)

Principle:

Study of the radical scavenger activity of the hydroxytyrosol dihydrosinapate of the invention of formula (IIa) and of the prior-art ferulic acid derivative in an acellular in vitro model, using DPPH.

Protocol:

1,1-Diphenyl-2-picrylhydrazyl, owing to its paramagnetic structure, is able to accept an electron or a hydrogen radical in order to become a stable diamagnetic molecule.

This free radical, which has a violet color in ethanol, exhibits a strong 530 nm absorption band.

The addition of a compound which provides electrons gives rise to decoloring of the 1,1-diphenyl-2-picrylhydrazyl in a manner proportional to the number of electrons accepted by the radical, and this decoloration can be monitored by measuring the absorbance at 530 nm (OD₅₃₀).

DPPH is incubated for 30 minutes in the presence of the test compound at a concentration of 10⁻⁵M, or on its own for the negative control. At the end of the incubation, the radical scavenger activity of the compound under test is evaluated by measuring the absorbance of the solution at 530 nm.

Results:

The results are presented as % inhibition of the DPPH radical, in Table 3a).

The radical scavenger activity of the test product is calculated as a percentage, according to the following formula:

100−((OD₅₃₀ in the presence of the compound under test/OD₅₃₀ in the absence of compound)×100)

TABLE 3a)
Percentage inhibitors of the DPPH radical,
obtained for hydroxytyrosol dihydrosinapate

| | | % inhibition of the |
|---|---|---|
| | Concentration tested | DPPH radical |
| Hydroxytyrosol | 1 μM | 10.7 +/− 0.59 |
| dihydrosinapate (IIa) | 5 μM | 59.68 +/− 0.89 |
| | 10 μM | 82.01 +/− 1.05 |
| | 30 μM | 86.68 +/− 0.44 |
| | 50 μM | 88.78 +/− 0.49 |
| | 100 μM | 88.28 +/− 0.56 |
| | 1000 μM | 89.14 +/− 0.40 |
| Prior-art ferulic acid | 1 μM | 6.02 +/− 0.758 |
| derivative | 5 μM | 35.02 +/− 1.209 |
| | 10 μM | 61.85 +/− 1.35 |
| | 30 μM | 89.91 +/− 0.66 |
| | 50 μM | 89.91 +/− 0.59 |
| | 100 μM | 89.91 +/− 0.66 |
| | 1000 μM | 90.99 +/− 0.46 |

### Example B.3b

### Study by the TBARS Test

Principle:

Study of the radical scavenger activity of the hydroxytyrosol dihydrosinapate of the invention of formula (IIa) and of the prior-art ferulic acid derivative by the TBARS test.

Protocol:

Two identical 96-well plates are produced according to a defined plate plan. 50 μL of a sample mixture prepared by diluting a solution of a test compound or of a negative control (vehicle medium without test compound) or of a positive control (D-L-alpha-tocopherol) to 1/10 in a liposomal suspension are distributed per well, in quadruplicate. One of the two plates is irradiated with UVB for 2 hours. The other plate, serving as a control, is incubated in the absence of light and at ambient temperature for 2 hours.

At the end of irradiation, an amount of 100 μL of 20% trichloroacetic acid solution was added per well, followed by an amount of 34 μL of 1% thiobarbituric acid solution. The plates are then incubated at 105° C. for 25 minutes.

The contents of each well are centrifuged at 5700 rpm for 5 minutes, and then an amount of 100 μL of supernatant was deposited in new plates, according to the same plan, and the absorbance was read off at 530 nm.

The positive experimental control was D-L-alpha-tocopherol, at a concentration of 1%, for which a percentage inhibition of 80% was obtained.

Results:

Table 3b) shows the results obtained, in % inhibition of lipid peroxidation, relative to the negative control.

TABLE 3b)
Percentage inhibitions of lipid peroxidation,
obtained for hydroxytyrosol dihydrosinapate

| | Concentration tested | |
|---|---|---|
| | (after dilution to 1/10th | |
| | in the liposomal | % inhibition of lipid |
| | suspension) | peroxidation |
| Hydroxytyrosol | 1 μM | −10.58 |
| dihydrosinapate (IIa) | 5 μM | 8.21 |
| | 30 μM | 7.14 |
| | 50 μM | 19.12 |
| | 100 μM | 41.95 |
| | 1000 μM | 77.04 |
| Prior-art ferulic acid | 1 μM | −16.95 |
| derivative | 5 μM | 6.69 |
| | 30 μM | 11.77 |
| | 50 μM | 31.74 |
| | 100 μM | 64.04 |
| | 1000 μM | 94.72 |

Conclusions from Example B.3a) and Example B.3b):

The test compound exhibits a substantial radical scavenger activity, even at low concentrations (10⁻⁶ M).

### Example B4

### Tests of Cytotoxicity

Principle:

Study of cell viability following application of the hydroxytyrosol dihydrosinapate of the invention of formula (IIa) to keratinocytes or melanocytes.

Protocol:

The cells (keratinocytes or melanocytes) were cultured in a 96-well plate at 37° C. under 5% CO2 in an appropriate medium until confluence. The medium was withdrawn and then an amount of 200 μL of a 0.5 mg/mL MTT solution was added. The plates were then incubated at 37° C. for 3 hours, after which the MTT solution was withdrawn from each well and replaced with DMSO. The plates were agitated for 15 minutes, and the optical density was measured at 550 nm.

The negative cytotoxicity control is PBS. The positive cytotoxicity control is 0.25% SDS. 0.1% DMSO was used as control.

Results:

Table 4a) shows the results of the percentage of viable cells thus obtained, where the cells are keratinocytes.

Table 4b) shows the results of the percentage of viable cells thus obtained, where the cells are melanocytes.

TABLE 4a)
Percentage cell viabilities obtained for hydroxytyrosol
dihydrosinapate on keratinocytes

| | | Standard |
|---|---|---|
| | Mean | deviation |
| PBS | 100.0 | 4.0 |
| SDS 0.25% | 1.3 | 0.1 |
| DMSO 0.1% | 93.6 | 5.0 |
| Hydroxytyrosol | 105.8 | 4.7 |
| dihydrosinapate (1 μM) | | |
| Hydroxytyrosol | 115.2 | 4.4 |
| dihydrosinapate (10 μM) | | |
| Hydroxytyrosol | 75.7 | 2.6 |
| dihydrosinapate (100 μM) | | |

TABLE 4b)
Percentage cell viabilities obtained for hydroxytyrosol
dihydrosinapate on melanocytes

| | | Standard |
|---|---|---|
| | Mean | deviation |
| T PBS | 100.0 | 2.83 |
| SDS 0.25% | 0.8 | 0.59 |
| DMSO 0.1% | 112.51 | 9.08 |
| Hydroxytyrosol | 109.67 | 7.63 |
| dihydrosinapate (1 μM) | | |
| Hydroxytyrosol | 107.53 | 8.95 |
| dihydrosinapate (10 μM) | | |
| Hydroxytyrosol | 111.30 | 3.50 |
| dihydrosinapate (100 μM) | | |

Conclusions from Example B.4a) and Example B.4b):

Hydroxytyrosol dihydrosinapate is not cytotoxic for concentrations of the order of 10⁻⁶, 10⁻⁵ and 10⁻⁴ M, in other words even for the higher concentrations tested, since the viability percentages obtained are greater than 75% viability (tolerated threshold).

### Example B5

### Tests of Antiinflammatory Activity

Principle:

Study of the antiinflammatory activity of the hydroxytyrosol dihydrosinapate of the invention of formula (IIa), by measurement of the inhibition of release of E2 prostaglandins by keratinocytes under the effect of UVB.

Protocol:

The keratinocytes from the A431 line are cultured in an appropriate medium at 37° C. for 72 hours under 5% CO2. The culture medium is then withdrawn and replaced with the medium containing the products under test, and also the positive and negative controls. The cells are subsequently irradiated with UV B at 30 mJ/cm2. The cells are incubated for an extra 24 hours at 37° C. under 5% CO2. The cell supernatants are subsequently recovered, and the E2 prostaglandins (PGE2) released are quantified by an ELISA assay.

An amount of 100 μL of each sample, control or PGE2 standard is transferred to the wells of a 96-well plate, which are coated with antibodies directed against rat immunoglobulin G (IgG). Then an amount of 25 μL of PGE2-peroxidase conjugate is added to each well, followed by an amount of 25 μL of anti-PGE2 monoclonal antibody. The plate is incubated at 4° C. overnight. The wells are then rinsed and an amount of 100 μL of peroxidase substrate is added. After 15 minutes of incubation at ambient temperature, the optical density is read off at 450 nm. The amount of prostaglandin E2 released after irradiation is deduced from a commercial PGE2 standard range from 0 to 400 pg/mL of PGE2.

The negative control, represented by the cells which were not treated with samples and which underwent irradiation by UV B, gave rise to 100% release of PGE2. The positive experimental control is aspirin, which at 0.03% gave rise to 5%+/−2% release of PGE2.

Results:

The results obtained with hydroxytyrosol dihydrosinapate (IIa) are as follows:

| Concentration of compound IIa | % release of PGE2 after irradiation |
|---|---|
| 30 μM | 22% |

Conclusion:

Hydroxytyrosol dihydrosinapate of formula (IIa) at 30 μM greatly inhibited prostaglandin E2 release by the keratinocytes after irradiation thereof with UVB. The compounds according to the invention therefore do have an antiinflammatory effect.

### C. Composition Examples

Using methods known to the skilled person, the different portions A, B, C, D, E, or F are mixed together to prepare a composition according to the present invention.

The term “products of the invention” refers to the compounds conforming to the general formula (I), preferably the compounds of formula (II), especially the preferred compounds conforming to the general formulae (IIa) or (IIb), and also mixtures thereof, and the amount thereof is adjusted such that the concentration thereof is approximately 1×10⁻⁴ M in the final composition.

### Example C1

### Use of the Products of the Invention on Cosmetic or Pharmaceutical Formulations of Oil-in-Water Emulsion Type

### Formulation 1a:

| | | |
|---|---|---|
| A | Water | qs 100 |
| | Butylene glycol | 2 |
| | Glycerin | 3 |
| | Sodium dihydroxycetyl | 2 |
| | phosphate, isopropyl | |
| | hydroxycetyl ether | |
| B | Glycol stearate SE | 14 |
| | Triisononanoin | 5 |
| | Octyl cocoate | 6 |
| C | Butylene glycol, methylparaben, | 2 |
| | ethylparaben, propylparaben, | |
| | pH adjusted to 5.5 | |
| D | Products of the invention | adjusted to 1 × 10⁻⁴M |

### Formulation 1b:

| | | |
|---|---|---|
| A | Water | qs 100 |
| | Butylene glycol | 2 |
| | Glycerin | 3 |
| | Polyacrylamide, isoparaffin, | 2.8 |
| | laureth-7 | |
| B | Butylene glycol, methylparaben, | 2 |
| | ethylparaben, propylparaben; | |
| | Phenoxyethanol, methylparaben, | 2 |
| | propylparaben, butylparaben, | |
| | ethylparaben | |
| | Butylene glycol | 0.5 |
| D | Products of the invention | adjusted to 1 × 10⁻⁴M |

### Formulation 1c:

| | | |
|---|---|---|
| A | Carbomer | 0.50 |
| | Propylene glycol | 3 |
| | Glycerol | 5 |
| | Water | qs 100 |
| B | Octyl cocoate | 5 |
| | Bisabolol | 0.30 |
| | Dimethicone | 0.30 |
| C | Sodium hydroxide | 1.60 |
| D | Phenoxyethanol, methylparaben, | 0.50 |
| | propylparaben, butylparaben, | |
| | ethylparaben | |
| E | Fragrance | 0.30 |
| F | Products of the invention | adjusted to 1 × 10⁻⁴M |

### Example C2

### Use of the Products of the Invention in a Water-in-Oil Formulation

| | | |
|---|---|---|
| A | PEG 30 dipolyhydroxystearate | 3 |
| | Capric triglycerides | 3 |
| | Cetearyl octanoate | 4 |
| | Dibutyl adipate | 3 |
| | Grapeseed oil | 1.5 |
| | Jojoba oil | 1.5 |
| | Phenoxyethanol, methylparaben, | 0.5 |
| | propylparaben, butylparaben, | |
| | ethylparaben | |
| B | Glycerin | 3 |
| | Butylene glycol | 3 |
| | Magnesium sulfate | 0.5 |
| | EDTA | 0.05 |
| | Water | qs 100 |
| C | Cyclomethicone | 1 |
| | Dimethicone | 1 |
| D | Fragrance | 0.3 |
| E | Products of the invention | adjusted to 1 × 10⁻⁴M |

### Example C3

Use of the Products of the Invention in a Shampoo or Shower Gel Formulation

| | | |
|---|---|---|
| A | Xanthan gum | 0.8 |
| | Water | qs 100 |
| B | Butylene glycol, methylparaben, | 0.5 |
| | ethylparaben, propylparaben | |
| | Phenoxyethanol, methylparaben, | 0.5 |
| | propylparaben, butylparaben, | |
| | ethylparaben | |
| C | Citric acid | 0.8 |
| D | Sodium laureth sulfate | 40.0 |
| E | Product of the invention | adjusted to 1 × 10⁻⁴M |

### Example C4

### Use of the Products of the Invention in a Lipstick Formulation and Other Anhydrous Products

| | | |
|---|---|---|
| A | Mineral wax | 17.0 |
| | Isostearyl isostearate | 31.5 |
| | Propylene glycol dipelargonate | 2.6 |
| | Propylene glycol isostearate | 1.7 |
| | PEG 8 beeswax | 3.0 |
| | Hydrogenated palm kernel oil | 3.4 |
| | glycerides, hydrogenated palm | |
| | glycerides | |
| | Lanolin oil | 3.4 |
| | Sesame oil | 1.7 |
| | Cetyl lactate | 1.7 |
| | Mineral oil, lanolin alcohol | 3.0 |
| B | Castor oil | qs 100 |
| | Titanium dioxide | 3.9 |
| | CI 15850:1 | 0.616 |
| | CI 45410:1 | 0.256 |
| | CI 19140:1 | 0.048 |
| | CI 77491 | 2.048 |
| C | Products of the invention | adjusted to 1 × 10⁻⁴M |

### Example C5

### Preparation of Pharmaceutical Formulations Containing the Product of the Invention

Formulation 5a:

| | Excipients | In g per tablet |
|---|---|---|
| A | Lactose | 0.359 |
| | Saccharose | 0.240 |
| B | Products of the invention | adjusted to 1 × 10⁻⁴M |

Formulation 5b:

| | Excipients | |
|---|---|---|
| A | Low-density polyethylene | 5.5 |
| | Liquid paraffin | qs 100 |
| B | Products of the invention | adjusted to 1 × 10⁻⁴M |

### Example C6

### Preparation of an Injectable Formula

| | Excipient | |
|---|---|---|
| A | Isotonic saline solution | 5 ml |
| B | Products of the invention | adjusted to 1 × 10⁻⁴M |

1.-20. (canceled)

21. A compound of general formula (I) below: 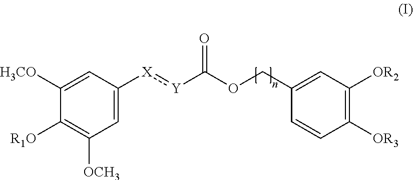
in which:
R₁, R₂ and R₃ independently of one another represent a hydrogen atom; a C1-12 alkyl group; a C2-12 alkenyl group; a C2-12 alkynyl group; a C3-12 cycloalkyl group; a C1-12 acyl group; a sulfonyl group or a phosphonate group; [symbol] represents a CH₂—CH₂ group or a CH═CH group;
n is an integer between 1 and 3;
or a salt thereof,
with the exception of the compound of formula (a) below:

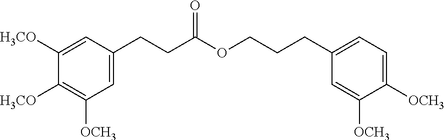 [SMILES: COc1ccc(CCCOC(=O)CCc2cc(OC)c(OC)c(OC)c2)cc1OC]

22. A compound as claimed in claim 21, wherein n is 2.

23. A compound as claimed in claim 21, wherein R₁, R₂ and R₃ independently of one another represent a hydrogen atom, a C1-C2 alkyl group, a sulfonyl group; or a phosphonate group; or a salt thereof.

24. A compound as claimed in claim 22, wherein R₁, R₂ and R₃ are identical and each represents a hydrogen atom.

25. A hydroxytyrosol dihydrosinapate of formula (IIa) below: 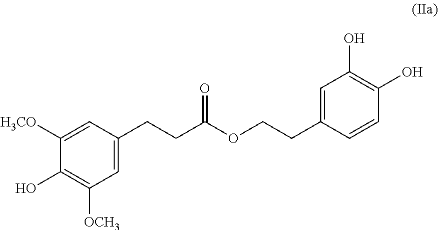 [SMILES: COc1cc(CCC(=O)OCCc2ccc(O)c(O)c2)cc(OC)c1O]
or a salt thereof.

26. A hydroxytyrosol sinapate of formula (IIb) below: 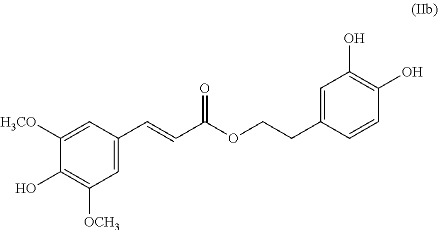 [SMILES: COc1cc(/C=C/C(=O)OCCc2ccc(O)c(O)c2)cc(OC)c1O]
or a salt thereof.

27. A depigmenting agent, and/or a radical scavenger comprising at least one compound as claimed in claim 21 or the compound of formula (a).

28. A method for preventing and/or combating skin aging, or preventing and/or combating at least one condition selected from the following: a dull and/or patchy complexion and/or a loss of radiance, and/or the transparency of the skin, and/or a loss of softness, and/or a loss of suppleness, and/or a loss of elasticity, and/or the premature formation of wrinkles and/or fine lines, and/or the appearance of pigment spots comprising the administration of an effective amount of at least one compound as claimed in claim 21 or of the compound of formula (a) 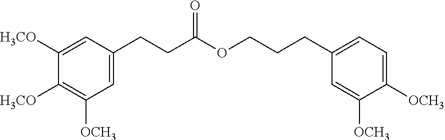 [SMILES: COc1ccc(CCCOC(=O)CCc2cc(OC)c(OC)c(OC)c2)cc1OC]
to a human being in need thereof.

29. A method for preventing and/or treating pathological hyperpigmentation and/or inflammation comprising the administration of an effective amount of a compound as claimed in claim 21 or of a compound of formula (a) 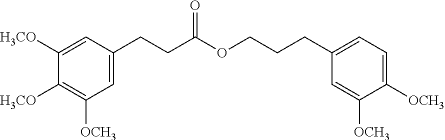 [SMILES: COc1ccc(CCCOC(=O)CCc2cc(OC)c(OC)c(OC)c2)cc1OC]
to a patient in need thereof.

30. A cosmetic and/or pharmaceutical composition comprising at least one compound as claimed in claim 21.

31. The composition as claimed in claim 30, wherein the compound is present at a concentration of between 1×10⁻⁶ and 1×10⁻¹ M.

32. The composition as claimed in claim 30, wherein the composition is intended for topical application.

33. The composition as claimed in claim 30, wherein the composition is intended for the skin of the hands, and/or of the face and/or of the neck and shoulders, and/or of the cranium, and/or for the nails, and/or the hair.

34. The cosmetic composition as claimed in claim 30, wherein the composition comprises at least one other ingredient of cosmetic interest, selected from: a depigmenting ingredient and/or an ingredient that enhances the radiance of the complexion, and/or radical scavenger ingredients.

35. A method of cosmetic care for preventing and/or combating nonpathological pigment spots and/or for preventing and/or combating aging-related pigment spots and/or pigment spots induced by exposure to the sun, and/or freckles, and/or postinflammation spots and/or spots which appear in response to aggressive influences, and/or spots of hormonal origin, and/or drug origin, and/or for preventing and/or combating pigment spots which are unesthetic manifestations of a pathology, and/or for whitening the skin and/or lightening the skin and/or imparting radiance and/or luminosity to the skin, and/or harmonizing the appearance of the skin, and/or for preventing and/or combating oxidative stress, and/or the benign manifestations caused by oxidative stress, the mucosae and/or the keratinous appendages, and/or for preventing and/or combating skin and/or mucosal aging, and/or the unesthetic and/or uncomfortable skin and/or mucosal manifestations caused by aggressive agents and/or aggressive conditions comprising the topical application of an effective amount of at least one compound as claimed in claim 21 or of the compound of formula (a) to a human being in need thereof.

36. A method of cosmetic care for preventing and/or combating nonpathological pigment spots and/or for preventing and/or combating aging-related pigment spots and/or pigment spots induced by exposure to the sun, and/or freckles, and/or postinflammation spots and/or spots which appear in response to aggressive influences, and/or spots of hormonal origin, and/or drug origin, and/or for preventing and/or combating pigment spots which are unesthetic manifestations of a pathology, and/or for whitening the skin and/or lightening the skin and/or imparting radiance and/or luminosity to the skin, and/or harmonizing the appearance of the skin, and/or for preventing and/or combating oxidative stress, and/or the benign manifestations caused by oxidative stress, the mucosae and/or the keratinous appendages, and/or for preventing and/or combating skin and/or mucosal aging, and/or the unesthetic and/or uncomfortable skin and/or mucosal manifestations caused by aggressive agents and/or aggressive conditions comprising the topical application of an effective amount of at least one of the cosmetic compositions as claimed in claim 32 to a human being in need thereof.

37. A process for synthesizing a compound as claimed in claim 21, comprising a step of coupling of an alcohol to an acid, the acid being sinapic acid and/or dihydrosinapic acid.

38. The synthesis process as claimed in claim 37, wherein dihydrosinapic acid is synthesized in situ from sinapic acid.

39. The synthesis process as claimed in claim 38, wherein the alcohol is hydroxytyrosol and the compound is the compound of formula (I) wherein n is 2.
